(19) United States
(12) Patent Application Publication
(10) Pub. No.: US 2008/0267899 A1
(43) Pub. Date: Oct. 30, 2008

(54) PLANT EXTRACT COMPOSITION FOR HAIR GROWTH

(75) Inventor: Hamit Leskaj, Montreal (CA)

Correspondence Address: BERESKIN AND PARR, 40 KING STREET WEST, BOX 401, TORONTO, ON, M5H 3Y2, CA

(73) Assignee: Mr. Hamit Leskaj, Montreal (CA)

(21) Appl. No.: 11/742,109

(22) Filed: Apr. 30, 2007

(51) Int. Cl. A61K 8/97; A61K 36/38; A61Q 7/00; A61K 36/63

(52) U.S. Cl. 424/74; 424/730; 424/771

(57) ABSTRACT

The present invention relates to a composition comprising a Hypericum perforatum flower extract in combination with a synergetic agent and uses thereof for hair growth.

### BACKGROUND OF THE INVENTION

(a) Field of the Invention

The present invention relates to a composition comprising a Hypericum perforatum flower extract in combination with a synergetic agent and uses thereof for hair growth.

(b) Description of Prior Art

Hair loss is a common problem, which occurs, for example, through natural processes or is often chemically promoted through the use of certain therapeutic drugs designed to alleviate conditions, such as cancer. Often such hair loss is accompanied by lack of hair regrowth, which causes partial or full baldness.

Hair growth occurs by a cycle of activity, which involves alternating periods of growth and rest. This cycle is often divided into three main stages, which are known as anagen, catagen and telogen. Anagen is the growth phase of the cycle and may be characterized by penetration of the hair follicle deep into the dermis with rapid proliferation of cells, which are differentiating to form hair. The next phase is catagen, which is a transitional stage marked by the cessation of cell division, and during which the hair follicle regresses through the dermis and hair growth is ceased. The next phase, telogen, is often characterized as the resting stage during which the regressed follicle contains a germ with tightly packed dermal papilla cells. At telogen, the initiation of a new anagen phase is caused by rapid cell proliferation in the germ, expansion of the dermal papilla, and elaboration of basement membrane components.

There have been many attempts in the literature to prevent the loss of hair or to invoke the regrowth of hair by, for example, the promotion or prolongation of anagen. Currently, there are two drugs approved by the United States Food and Drug Administration for the treatment of male pattern baldness: topical minoxidil (marketed as ROGAINE™ by Pharmacia) and oral finasteride (marketed as PROPECIA™ by Merck & Co., Inc.). For several reasons, however, including safety concerns and/or limited efficacy, the search for efficacious hair growth inducers is ongoing.

U.S. Pat. No. 6,113,907 entitled “Pharmaceutical grade StJohn's Worth” describes the processing and evaluation of the bioactivity of St. John's Wort (Hypericum perforatum) materials to produce drug preparations. This patent mainly orientates the use of the produced drugs to the alleviation of mild to moderate depression and headache, including tension headaches, drugs that have anti-viral and anti-microbial indications as well as being useful to promote wound or burn healing. This patent addresses the production of drugs developed with various parts of the Hypericum perforatum plant and no demonstration of any hair related usages for Hypericum perforatum derived materials are disclosed.

U.S. Pat. No. 7,195,783 entitled “Hypericin and hypericum extract: specific T-type calcium channel blocker, and their use as T-type calcium channel targeted therapeutics” describes a method of treating a patient suffering from chronic heart failure, congestive heart failure, ischemic condition, arrythmia, hypoinsulinemia, hyperinsulinemia, hyperaldosteronemia, epilepsy or preterm through administration of pure hypericin or hypericin-containing Hypericum perforatum extract. The pure hypericin or hypericin-containing Hypericum perforatum extract inhibits T-type calcium channels in a patient's cells and therefore allows treatment of the described diseases or disorders. There is no correlation between the use of any Hypericum perforatum extracts and any topical purposes including hair loss.

United States Patent Application No. 20040009142 entitled “Synergistically active mixture which inhibits hair growth” describes a process for inhibiting hair growth on skin comprising contacting it with a composition comprising hydrolyzed soya proteins, one plant extract selected from the group consisting of Hypericum perforatum, Hamamelis virginiana, Arnica Montana, Salix alba and mixtures thereof, and optionally an auxiliary component selected from urea, menthol, propylene glycol, salicylic acid and mixtures thereof The Hypericum perforatum plant extract consists of both extract of the whole plant and to extracts of parts of the plant and mixtures thereof. The Hypericum perforatum extract of this patent is always used in correlation with hydrolyzed soya proteins and with other components in preferred embodiments. However, Hypericum perforatum extract and its uses for hair growth are not addressed through this patent.

U.S. Pat. No. 6,428,820 entitled “Extracts of Hypericum perforatum and formulations containing them” relates to a method for extracting Hypericum perforatum by fractioning water-alcohol, alcohol extracts of the plant with esthers of water-immiscible C₁-C₅ alcohols and to formulations containing the extract. The method for the extraction of Hypericum perforatum of this patent is limited to the use of alcohol or acetone solvents with various Hypericum perforatum plant parts. The use of a synergetic agent for the preparation of an extract of Hypericum perforatum flowers is not addressed through this patent nor its potential use for hair growth.

International Patent No. WO 2005099728 entitled “Pharmaceutical compositions from Beard Lichen (Usnea Barbata) and St-John's Wort (Hypericum perforatum) and their use” describes a pharmaceutical composition made from Usnea barbata and its use in the treatment of skin diseases such as various forms of dermatitis, and skin aging, and for the treatment of skin infections with Pityrosporon yeast fungi, acne and rosacea. A preferred composition is the combination with an extract from Hypericum perforatum. However, there is no correlation between the use of an extract of Hypericum perforatum and potential hair growth stimulation properties.

It would be highly desirable to be provided with a natural, non-synthetic composition of Hypericum perforatum flower extract in combination with a synergetic agent for improving hair growth in a subject.

### SUMMARY OF THE INVENTION

In accordance with the present invention, it has been found that topical administration of Hypericum perforatum flower particles in combination with a synergetic agent can be used to increase hair growth in patients suffering from hair loss, or to prevent or delay hair loss in patients just beginning to lose their hair.

In accordance with an embodiment of the present invention, there is provided a composition comprising Hypericum perforatum flower particles in combination with a synergetic agent.

The Hypericum perforatum flower particles may be from sepals, petals, stamens and/or pistils.

The synergetic agent may be chosen from olive oil, almond oil, shea butter derived oil, aloe oil and colloidal oat suspension.

The olive oil synergetic agent may be chosen from extra-virgin olive oil, virgin olive oil, olive oil and olive-pomace oil.

In accordance with still another embodiment of the present invention, there is provided a topical composition for improving hair growth comprising an effective amount of the composition of Hypericum perforatum flower particles in combination with a synergetic agent in association with a suitable topical carrier.

The suitable carrier may be a cream, a shampoo, a gel or a lotion.

The Hypericum perforatum flower particles may be present in an amount from about 80 g. to about 120 g. in 1 L of the composition.

More preferably, the Hypericum perforatum flower particles may be present in a 100 g. dosage in 1 L of the composition.

The synergetic agent may be present in a 800 ml to 1200 ml dosage in the composition.

More preferably, the synergetic agent is present in a 1000 ml dosage in the composition.

In accordance with still another embodiment of the present invention, there is provided a method for the preparation of the composition of Hypericum perforatum flower particles in combination with a synergetic agent of the present invention, said method comprising:
a) mixing particles of a Hypericum perforatum flower with a synergetic amount of a synergetic agent; and
b) storing the mixture of step a) in a container for a sufficient period of time for said flower particles to macerate at a pharmaceutically acceptable temperature.

The Hypericum perforatum flower particles may be obtained by chopping, mincing, cutting, stripping and grinding the flower.

The sufficient period of time for the flower particles to macerate may consist of between 21 to 30 days.

The pharmaceutically acceptable temperature may be between 18° C. and 20° C.

The container may be stored without direct sunlight or in the dark.

In accordance with still another embodiment of the present invention, there is provided a method for the preparation of the topical composition of the present invention, the method comprising the steps of mixing a sufficient amount of the composition of the present invention in combination with a suitable carrier.

In accordance with still another embodiment of the present invention, there is provided a method for the treatment of hair loss in a subject, said method comprising the steps of topically contacting a region presenting hair follicles with a sufficient amount of the composition of the present invention and massaging the composition to allow penetration in the skin, that more preferably may be deprived of hair.

The method may include topically contacting a region presenting hair follicles with a sufficient amount of the composition, which consists in contacting the skin and hair of the region.

More preferably, the region may be a region in which hair growth has diminished.

More preferably, the region may be a region in which hair loss has increased.

More preferably, the region may be a region in which hair growth has diminished and hair loss has increased.

The method may include topically contacting the region with the composition 3 to 4 times a day for a 3 to 4 concurrent days treatment period in the case of a curative treatment.

More preferably, the region may remain unwashed during the 3 to 4 days treatment period.

More preferably, the region may be cleaned with an organic soap and/or shampoo during the 3 to 4 days treatment period.

The method may include topically contacting the region with the composition every night for a preventive treatment.

The method may include massaging the composition on the region for at least one minute.

In accordance with still a preferred embodiment of the present invention, there is provided the use of a composition of Hypericum perforatum flower particles in combination with a synergetic agent for the manufacture of a topical medicament for the treatment of hair loss in a subject.

More preferably, the topical administration of the medicament may include topically contacting a region presenting hair follicles with a sufficient amount of the composition and massaging the composition to allow penetration in the skin, which preferably may be deprived of hair.

All references referred herein are hereby incorporated by reference.

### DETAILED DESCRIPTION OF THE INVENTION

In accordance with the present invention, it has been found that topical administration of Hypericum perforatum flower particles in combination with a synergetic agent can be used to increase hair growth in patients suffering from hair loss, or to prevent or delay hair loss in patients just beginning to lose their hair.

The first recorded use of Hypericum perforatum extract for medicinal purposes dates back to ancient Greece, and has been used ever since. Hypericum perforatum was also used by Native Americans internally as an abortifacient and externally as an anti-inflammatory, astringent, and antiseptic. In modern medicine, standardized Hypericum perforatum extract is commonly used as a treatment for depression and anxiety disorders. However, the benefits of a composition of Hypericum perforatum for hair growth purposes have not been addressed in the past. The present invention specifically aims at the demonstration of the important hair growth stimulation and hair loss reduction properties of a topical application of Hypericum perforatum flower particles in combination with a synergetic agent.

The two main components of the composition of the present invention consist of Hypericum perforatum flower particles and a synergetic agent. The Hypericum perforatum flower particles comprise particles of the sepals, petals, stamens and pistils enclosing active ingredients that operate concurrently with the synergetic agents in which they are incorporated to promote important hair growth and hair loss diminution. Synergetic agents used in the composition consist of various types of oils having very high hydrating and skin healing properties. The combination of those oils with the active ingredients of the Hypericum perforatum flower particles creates a synergetic effect on hair follicles that has been brought to light through this invention. Indeed, topical application of the composition has been proven to be highly effective in increasing hair growth in patients suffering from hair loss, or in preventing or delaying hair loss in patients just beginning to lose their hair through repeated topical application in the region to treat. The preferred formulation of the composition resides in the combination of Hypericum perforatum flower particles with olive oil and the preferred method of treatment consists of 3 to 4 daily applications of the composition over 3 to 4 subsequent days without cleaning of the treated region. In both 3 to 4 days curative or nightly preventive treatment, the composition of the present invention has been proven to reinforce hair strength, brightness and general health, therefore decreasing hair loss, as well as inducing stimulation of hair follicles, resulting in subsequent hair growth. Moreover, the composition has the quality of being highly natural and non-synthetic, therefore generating no side effects on the user.

The present invention will be more readily understood by referring to the following examples which are given to illustrate the invention rather than to limit its scope.

### EXAMPLE 1

### Hypericum perforatum Flower Particles and Olive Oil Composition

Preparation of the compositon. 100 g of Hypericum perforatum flower particles were prepared through stripping of the flowers sepals, petals, stamens and/or pistils. The Hypericum perforatum flower particles are then mixed with 1 L of olive oil. The composition is introduced in glass container which lid is closed. The composition is stored without direct sunlight at a temperature of about 18° C. to 20° C. and for a period of time of about 21 to 30 days.

Posology: The composition is applied 3 to 4 times a day on the region to treat in an amount sufficient to allow good penetration of the product in the hair roots and in the surrounding skin. The composition is massaged on the region to treat for at least 1 minute. It is indicated to avoid washing the treated region for the 3 to 4 days treatment period. If cleaning is necessary, organic shampoos or soaps should be used rather than chemical ones.

Results. The results are presented in Table 1. The positive response observed in subjects treated with the composition of the present invention constitutes direct evidence that the product is effective in increasing hair growth in patients suffering from hair loss and preventing or delaying hair loss in patients just beginning to lose their hair.

TABLE 1
Results of the treatment of hair loss and promotion of hair growth in
11 subjects with the composition of Hypericum perforatum and olive oil.

| Subject | Sex | Age | Treatment term | Results |
|---|---|---|---|---|
| 001 | F | 45 | 2 months | ++++ |
| 002 | M | 32 | 5 weeks | +++ |
| 003 | M | 38 | 3 months | +++ |
| 004 | M | 59 | 1 month | +++ |
| 005 | F | 34 | 2 months | +++ |
| 006 | F | 53 | 4 months | +++ |
| 007 | M | 72 | 3 months | +++ |
| 008 | M | 58 | 6 months | ++++ |
| 009 | M | 45 | 4 months | +++ |
| 010 | M | 35 | 1^{1/2} months | +++ |
| 011 | M | 29 | 3^{1/2} months | +++ |

++++ = discontinuation of hair loss and important hair growth
+++ = discontinuation of hair loss and hair growth
++ = discontinuation of hair loss
+ = decrease in hair loss

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition comprising Hypericum perforatum flower particles in combination with a synergetic agent.

2. The composition according to claim 1, wherein said particles of the Hypericum perforatum flower consists of sepals, petals, stamens and/or pistils.

3. The composition according to claim 1, wherein said synergetic agent is chosen from olive oil, almond oil, shea butter derived oil, aloe oil and colloidal oat suspension.

4. The composition according to claim 1, wherein said synergetic agent is olive oil.

5. The composition according to claim 4, wherein said olive oil is extra-virgin olive oil, virgin olive oil, olive oil and olive-pomace oil.

6. A topical composition for improving hair growth comprising an effective amount of said composition of claim 5 in association with a suitable topical carrier.

7. The topical composition according to claim 6, wherein said suitable carrier is a cream, a shampoo, a gel or a lotion.

8. A method for the preparation of the composition of claim 5, said method comprising the steps of:
a) mixing Hypericum perforatum flower particles with a synergetic amount of a synergetic agent; and
b) storing the mixture of step a) in a container for a sufficient period of time for said flower particles to macerate at a pharmaceutically acceptable temperature.

9. The method according to claim 8, wherein said Hypericum perforatum flower particles consists of petals, sepals, stamen and/or pistils.

10. The method according to claim 9, wherein said Hypericum perforatum flower particles are obtained by chopping, mincing, cutting, stripping and grinding said flower.

11. The method according to claim 9, wherein said Hypericum perforatum flower particles are present in an amount from about 80 g to about 120 g in 1 L of the composition.

12. The method according to claim 11, wherein said Hypericum perforatum flower particles are present in a 100 g dosage in 1 L of the composition.

13. The method according to claim 8, wherein said synergetic agent is olive oil, almond oil, shea butter derived oil, aloe oil and colloidal oat suspension.

14. The method according to claim 8, wherein said synergetic agent is olive oil.

15. The method according to claim 14, wherein said olive oil is extra-virgin olive oil, virgin olive oil, olive oil and olive-pomace oil.

16. The method according to claim 8, wherein said synergetic agent is present in a 800 ml to 1200 ml dosage in the composition.

17. The method according to claim 16, wherein said synergetic agent is present in a 1000 ml dosage in the composition.

18. The method according to claim 8, wherein said sufficient period of time consists of between 21 to 30 days.

19. The method according to claim 8, wherein said pharmaceutically acceptable temperature is between 18° C. and 20° C.

20. The method according to claim 8, wherein said container is stored without direct sunlight or in the dark.

21. A method for the preparation of the topical composition of claim 6, said method comprising the steps of mixing a sufficient amount of the composition of claims 1 to 5 in combination with a suitable carrier.

22. The method according to claim 21, wherein said suitable carrier is a cream, a shampoo, a gel, a lotion.

23. A method for the treatment of hair loss in a subject, said method comprising the steps of topically contacting a region presenting hair follicles with a sufficient amount of the composition of claim 6 and massaging the composition to allow penetration in the skin.
